(19) United States
(12) Patent Application Publication
(10) Pub. No.: US 2006/0140860 A1
(43) Pub. Date: Jun. 29, 2006

(54) Computational knowledge model to discover molecular causes and treatment of diabetes mellitus

(75) Inventor: Jack Pollard JR., Somerville, MA (US)

Correspondence Address: KIRKPATRICK & LOCKHART NICHOLSON GRAHAM LLP, STATE STREET FINANCIAL CENTER, ONE LINCOLN STREET, BOSTON, MA, 02111-2950, US

(73) Assignee: Genstruct, Inc., Cambridge, MA (US)

(21) Appl. No.: 11/294,251

(22) Filed: Dec. 5, 2005

Related U.S. Application Data
(60) Provisional application No. 60/634,405, filed on Dec. 8, 2004.
(60) Provisional application No. 60/692,509, filed on Jun. 21, 2005.

(51) Int. Cl. A61K 49/00; G01N 33/567

(52) U.S. Cl. 424/9.1; 435/7.2

(57) ABSTRACT

In one aspect, the invention comprises a method of developing an immunosuppressant drug with reduced propensity to induce type II diabetes mellitus symptoms by assaying candidate molecular entities for binding preferentially to leukocyte isoform calcineurin and less preferentially to muscle cell isoform calcineurin. In another aspect, the invention comprises a method for determining the onset, severity, or response to treatment of post-transplant diabetes mellitus comprising determining from a patient at risk of contracting post-transplant diabetes mellitus, at least two of increased expression of HDAC5 protein; increased expression of HNF4A protein; decreased expression of NRF1 protein; decreased expression of PPARGC1 protein; and decreased expression of PPP3CA protein.

### RELATED APPLICATIONS

This application claims priority to U.S. Provisional Patent Application Ser. No. 60/634,405, filed Dec. 8, 2004, and U.S. Provisional Patent Application Ser. No. 60/692,509, filed Jun. 21, 2005, the entire disclosures of which are herein incorporated by reference.

### BACKGROUND OF THE INVENTION

Type II diabetes mellitus (DM2) is a complex and multigenic disease. Our understanding of the mechanisms of its pathophysiology and corresponding therapeutic interventions is limited. While the human genome sequence and genome-wide profiling technologies have facilitated system-level measurements, methods to interpret these measurements into models of discrete signaling, metabolic, and gene regulatory mechanisms have lagged behind. Accordingly, a system-level approach to measuring and modeling the multiple variables associated with DM2 is necessary to improve our understanding of this disease and treatment options.

Calcineurin is a heterodimeric calcium and calmodulin-dependent serine-threonine protein phosphatase consisting of a catalytic A subunit and a regulatory calcium-binding B subunit. The calcineurin inhibitors tacrolimus (FK506) and cyclosporine A (CsA) are essential immunosuppressive drugs for the clinical management of rejection in organ transplantation. While calcineurin is widely distributed throughout the body, including the brain, heart, liver, kidney, pancreas and skeletal muscle, the rationale for the use of calcineurin in transplant rejection protocols has been the targeting of leukocyte-associated calcineurin as a means to suppress leukocyte function and prolong graft survival. However, immunosuppressive therapy with these inhibitors represents a significant independent risk factor for the development of post-transplant diabetes mellitus (a category of type II diabetes mellitus) and post-transplant diabetes mellitus itself significantly compromises graft and patient survival.

Clinical management of post-transplant diabetes mellitus involves assessment of predisposing risk factors such as age, family history, ethnic background, obesity and immunosuppressive protocol in an attempt to minimize the overall risk for development of new-onset disease. However, the pathogenesis of diabetes and impaired glucose tolerance secondary to therapy with calcineurin inhibitors is unknown, and the risk for development of post-transplant diabetes mellitus can not be currently predicted.

### SUMMARY OF THE INVENTION

The present invention exploits the discovery that the activity or level of skeletal muscle calcineurin can be used to predict the development of new-onset post-transplantation diabetes mellitus. This discovery can also be used to screen disease progression and conversion of a pre-diabetic state to an overt diabetic state during the course of immunosuppressive therapy, to monitor the pharmacologic effects of calcineurin inhibition as a surrogate of glucose tolerance, and to design individualized pre and post-transplant immunosuppressive protocols to minimize drug-induced new-onset diabetes.

In one aspect, the invention provides a method of developing an immunosuppressant drug with reduced propensity to induce type II diabetes symptoms. The method comprises assaying candidate molecular entities for binding preferentially to leukocyte isoform calcineurin and less preferentially to muscle cell isoform calcineurin.

According to one embodiment of the method of developing an immunosuppressant drug, the candidate molecular entities assayed according to the method comprise molecules adjacent in chemical space to FK506 or a cyclosporine. In another embodiment, the binding assay is conducted using immobilized calcineurin isoforms or labeled soluble calcineurin isoforms.

In one embodiment according to the method of developing an immunosuppressant drug, the leukocyte isoform calcineurin is an (A-A, B-B, A-B) calcineurin dimer. In another embodiment, the muscle cell isoform calcineurin is an (A-A, B-B, A-B) calcineurin dimer. In yet another embodiment, the leukocyte isoform calcineurin and the muscle cell isoform calcineurin are splice variants of each other.

In another aspect, the invention is a method for determining the onset, severity, progression or response to treatment of post-transplantation diabetes mellitus, comprising determining from a patient at risk of contracting post-transplant diabetes mellitus, at least two of increased expression or activity of HDAC5 protein, increased expression or activity of HNF4A protein, decreased expression or activity of NRF1 protein, decreased expression or activity of PPARGC1 protein, decreased expression or activity of PPP3CA protein, and decreased expression or activity of calcineurin proteins.

In one embodiment according to the method of determining onset, the method comprises determining decreased expression or activity of PPARGC1 protein. In another embodiment, the method comprises assaying for protein concentrations or activity in a muscle cell from a muscle biopsy from a said patient. In yet another embodiment, the method comprises assaying for gene transcripts or biomolecules produced by interaction with a said protein as a proxy for an increase or decrease in said protein expression or activity.

### BRIEF DESCRIPTION OF THE DRAWINGS

FIG. 1 is an illustrative embodiment of a causal model.

FIG. 2 is an illustrative embodiment of a casual model including reverse and forward causal analysis.

FIG. 3 is an illustrative embodiment of a causal model predicting the influence of the thiazolidinedione class of drugs.

FIG. 4A is an illustrative embodiment of a forward causal analysis following a perturbation to the causal model.

FIG. 4B is an illustrative embodiment of one effect of the perturbation illustrated in FIG. 4A.

FIG. 5 is an illustrative embodiment of the intersection of two causal analyses.

### DETAILED DESCRIPTION OF THE INVENTION

A system-level approach to measuring and modeling the multiple variables associated with type II diabetes mellitus (DM2) has been developed to improve our understanding of DM2 and possible treatment options. A causal model of gene regulation in human skeletal muscle was developed by integrating genome-wide profiling measurements with system-level models of molecular cause-and-effect relationships. Using computer-aided causal reasoning applications, the casual model was probed to discover mechanisms causally linked to altered expression profiles in DM2 to define discrete mechanisms of gene regulation in skeletal muscle biopsies from DM2 patients. The resulting hypotheses describe biologic effects in DM2 and enable assessment of molecular targeted diagnostic and therapeutic tools.

The development of post-transplant diabetes mellitus threatens the clinical outcome of transplantation and patient survival, and its complications result in greater health care costs post-organ transplantation. Causal analysis of the human skeletal muscle model implicates skeletal muscle PPP3CA (skeletal muscle calcineurin) activity and/or level as a risk factor for the pathogenesis of post-transplant diabetes mellitus. Forward causal analysis simulations accurately predicted and confirmed changes previously observed in diabetic subjects by transcript profiling, including the coordinate reduction of PGC1 and NERF1-dependent genes involved in oxidative phosphorylation. Reverse causal analysis simulations showed that coordinated inhibition of skeletal muscle PPP3CA and MEF2A causally predicted insulin resistance and hyperglycemia, as evidenced by downregulation of insulin receptor (IRS1) and glucose transport (GLUT4; SLC2A4) activity. Because calcineurin (PPP3CA) is the therapeutic target of tacrolimus (FK506) and cyclosporine A (CsA), these findings provide a direct and previously unrecognized linkage between skeletal muscle PPP3CA expression and glucose tolerance.

Thus, the invention involves assessment of skeletal muscle calcineurin expression by transcript profiling or proteomic methods, and correlation of calcineurin activity data with post-transplant diabetes mellitus, either together with or independent of other predisposing factors. Assessments may be used, among other things, to predict the development of new-onset diabetes post-transplantation, to screen disease progression and conversion of a pre-diabetic to an overt diabetic state during the course of immunosuppressive therapy, to monitor therapeutically the pharmacologic effects of calcineurin inhibition as a surrogate of glucose tolerance, and to design individualized pre-transplant and post-transplant immunosuppressive protocols to minimize drug-induced new-onset diabetes.

The incidence of post-transplant diabetes mellitus is higher in patients treated with tacrolimus than cyclosporine (odds ratio 5.03, 95% CI 2.04-12.36), although the use of tacrolimus is associated with fewer (odds ratio compared to cyclosporine 0.52) episodes of acute rejection attributable to its 10-100 fold greater immunosuppressive activity. Through simulation analyses, described in detail below, therapeutic intervention resulting in inhibition of calcineurin recapitulated the gene expression network described for diabetic subjects.

Thus, the invention provides a theoretical framework for the development of a tool to regulate skeletal muscle PPP3CA by drugs, to develop High Throughput Screening (HTS) assays, and to develop methods for the diagnosis and treatment of the disease in humans.

Because calcineurin isoforms may be differentially expressed, development of small molecules with increased potency and selectivity against leukocyte calcineurin than skeletal muscle calcineurin should result in significant clinical benefit post-transplantation by limiting muscle PPP3CA targeting, and thus, decreasing the frequency of development or severity of post-transplant diabetes mellitus.

Causal Modeling in Biology

A model that describes the influence exerted by one component in a system, for example, protein abundance or activity, on another component is called a causal model. Causal models provide a statistical framework to infer causes for changes in the system's components as the system transitions between states. For example, the model can infer potential causes for changes in the muscle gene expression profiles in type II diabetes mellitus (DM2). Causal models also provide a statistical framework to infer effects on the components resulting from system perturbations. For example, the model can infer which muscle gene expression changes are attributable only to increased insulin levels.

The exemplary modeling and analysis techniques described in this invention are aimed at facilitating inference on the causes for and effects of, for example, the changes in RNA expression levels. The analysis techniques require leveraging prior knowledge to gain efficiency and resolution over purely statistical approaches. Therefore, the exemplary techniques of the invention employ an ontology to structure and render computable knowledge about the causes for changes in activities and abundance of the components in the muscle and the effects those causes engender. At the highest level, the ontology describes the measurable components in molecular biology. For example, measurable molecular biology components may include protein abundance, catalytic activities and biological processes. The ontology also describes the relationships between the components. Relationships between the components may be described as associative or causal and may include the sign or direction of the influence. The ontology also describes the biological context of the components and their relationships by linking them to specific organisms, tissues, cell types, subcellular compartments, or other biological categories.

The causal analysis algorithms use as variables prior knowledge in the form of a causal model and a set of changes in the components of a system profiled in different states. For example, changes in the system may include changes in the muscle gene expression profiles comparing the DM2 state with the normal state. In the case of RNA expression data, reverse causal analysis interrogates the model to find immediate upstream transcriptional controllers. Exemplary upstream transcriptional controllers include the abundance or activity of transcription factors, co-activators, co-repressors, or modulators of transcript stability, whose increase or decrease hypothetically could be a cause of the observed changes in the RNA profiles. Once the immediate upstream controllers are located, forward causal analysis scores each hypothetical cause by comparing the predicted RNA profile with the observed profile.

Causal Model of Human Skeletal Muscle Biology and DM2

Metabolic abnormalities associated with DM2 are in part caused by changes in transcriptional regulatory networks of skeletal muscle. Two recent, independent transcript profile studies of human skeletal muscle biopsies from DM2 patients have shown the coordinated down-regulation in genes associated with oxidative phosphorylation and ATP biosynthesis and genes expressed in the mitochondria, collectively called the OXPHOS genes. These studies explain altered OXPHOS transcription by regulation of the transcriptional activity of NRF1 and PPARGC1a. However, discrete mechanisms that link OXPHOS transcription to both its downstream effects and its upstream causes need to be defined in order to determine if altered OXPHOS transcription is a cause or an effect of the characteristics of DM2 such as impaired insulin signaling.

An exemplary model was created for human skeletal muscle biology and DM2 containing more than 157,000 molecular components, including, for example, genes, proteins, metabolites and pathways, and more than 210,000 relationships between those components. Of those relationships, more than 24,000 are causal relationships. For example, the exemplary human skeletal muscle biology and DM2 model describes the molecular effects of increased insulin, fatty acids and glucose on muscle as well as many other causes for changes in gene expression including glucose transport, lipid metabolism, insulin signaling, glucose oxidation, and glucosamine metabolism.

The generated model can be analyzed to elucidate relevant relationships and causal changes. One exemplary causal change is an RNA expression state change. For example, using the exemplary model of human skeletal muscle biology and DM2, the results of numerous studies related to human skeletal muscle and DM2 were compared and separately analyzed using standard statistical packages. One exemplary analysis compared RNA expression profiles of human skeletal muscle biopsies from DM2 patients with family history-negative control subjects from a Mexican-American study. Another exemplary analysis compared DM2 patients with normal glucose tolerance control subjects from a Scandinavian study. Following statistical analysis, raw intensities were normalized within each array with locally weighted linear regression (loess), low-intensity signals were filtered, and global scaling was performed on each array to make the intensities between the DM2 patients and control subjects comparable within each study.

Separate differential expression analyses can be performed on the normalized data from each study. Using the exemplary model, a gene-by-gene analysis of variance was performed comparing DM2 patients with control subjects. Genes differentially expressed in two or more comparisons between the DM2 and the control subjects were used to classify the samples in the respective studies. In the exemplary model, only fourteen genes were consistently modulated in both studies. Using over-representation analysis, both the original studies and the exemplary model analysis confirmed that OXPHOS genes are present in the modulated set of genes at disproportionately high frequencies. Because the exemplary causal model is qualitative in nature, the expression changes were categorized before they were used in causal analysis. The expression changes were then divided into three categories: genes that are increased, decreased, or unchanged in DM2 muscle compared with control.

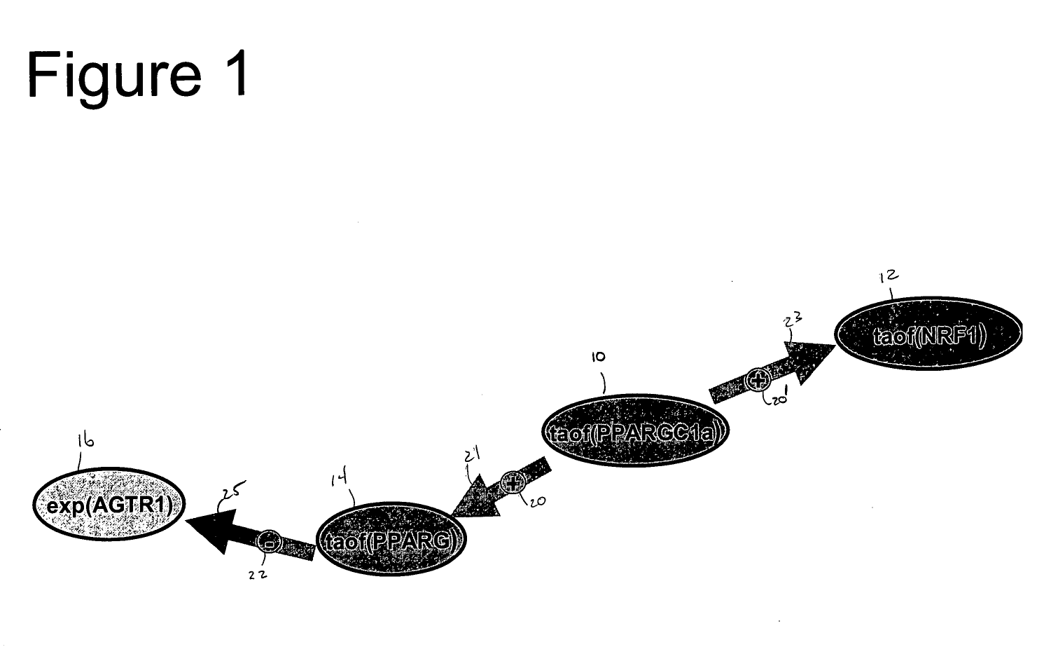

Causal models are computationally attractive tools to determine the causes and effect of a change within a biological system because inferencing can be automated and the model rendered as a directed graph. FIG. 1 is an exemplary directed graph of one embodiment of a causal model representing the influence of the transcriptional activity of the transcriptional co-activator PPARGC1a on the transcriptional activity of PPARG and NRF1. With reference to FIG. 1, the transcriptional activity of PPARG 14 and NRF1 12 as well as the transcriptional activity of the transcriptional co-activator PPARGC1a 10 are represented as nodes. Increases in transcriptional activity are indicated as nodes with light shading, for example, the expression level of AGTR1 16 is asserted to increase. Decreases in transcriptional activity are indicated as nodes with dark shading, for example, the transcriptional activity of PPARG 14, PPARGC1a 10 and NRF1 12 are all asserted to decrease. The edges are represented as arrows, for example 21, 23, 25, and the directionality of the edges is represented as either a plus (+) 20 or a minus (−) 22 sign.

With continued reference to FIG. 1, the edges of the directed graph assert that the transcriptional activity of PPARGC1a 10 positively 20 influences 21 the transcriptional activity of PPARG 14 and also positively 20′ influences 23 the transcriptional activity of NRF1 12. The edges also assert that the transcriptional activity of PPARG 14 negatively 22 influences 25 the expression level of AGTR1 16. Additionally, the influence of indirectly connected nodes can be identified by tracing connecting paths and multiplying the signs of the intervening edges, as in the case of the expression level of AGTR1 16.

The exemplary causal model represented in FIG. 1 is qualitative in nature in that it describes only the vector of influence that PARGC1a's transcriptional activity 10 exerts on PPARG 14 and NRF1 12 transcriptional activity. The qualitative causal model does not describe the magnitude of the influence or how multiple influences should be integrated. Qualitative causal models occupy a tractable middle ground in a taxonomy of models between more abstract association models that cannot be used to infer causes or effects but require no prior knowledge and more detailed quantitative models that can be used to infer causes and effects but require very detailed knowledge such as diffusion or rate constants in a specific sub-cellular environments.

Causal Analysis of the Human Skeletal Muscle Biology and DM2 Model

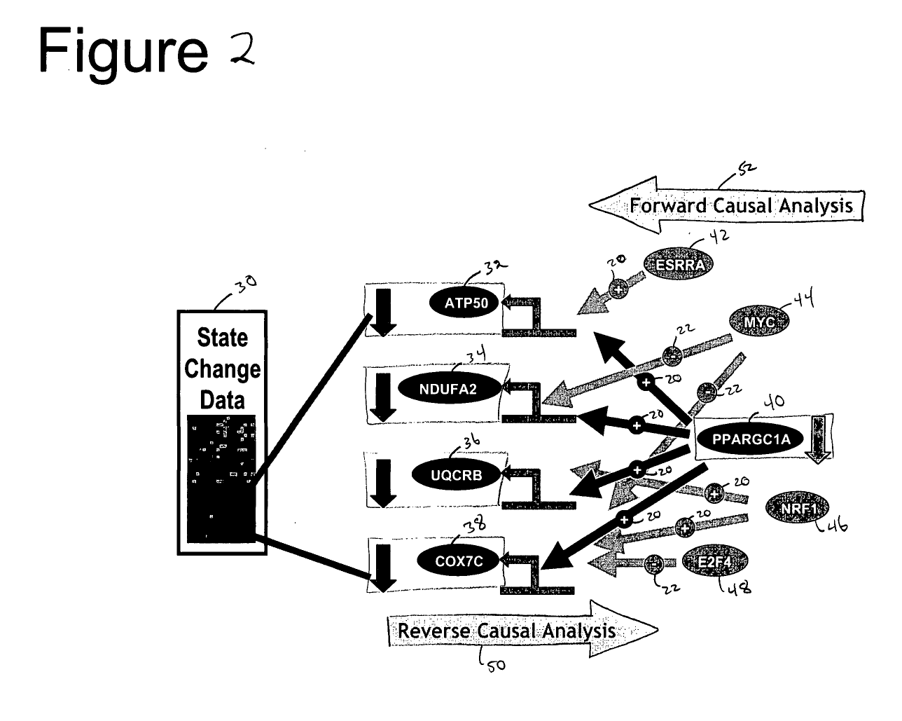

FIG. 2 is an exemplary directed graph representing reverse and forward causal analysis of the state change data of the OXPHOS genes ATP50 32, NDUFA2 34, UQCRB 36, and COX7C 38 conducted within the exemplary human skeletal muscle and DM2 model. Reverse causal analysis 50 of the human skeletal muscle and DM2 model predicts five transcriptional modifiers that could account for the observed changes in the RNA profiles of the OXPHOS genes: ESRRA 42, MYC 44, PPARGC1a 40, NRF1 46, and E2F4 48 (light shaded arrows). Forward causal analysis 52 predicts that only a decrease in the transcriptional activity of PPARGC1a 40 can explain all of the observed changes with no contradictions (dark shaded arrows).

Each hypothesis predicted by causal analysis is scored according to two probabilistic scoring metrics that examine orthogonal aspects of the probability of a hypothetical cause explaining a given number of state changes: richness and concordance. Richness is the probability that the number of observed changes that match the directionality, for example, increased or decreased abundance, of the changes predicted by the model could have occurred by chance alone. Only hypotheses that pass preset metric thresholds are used as inputs for continued upstream exploration in the model in progressive cycles of reverse and forward causal analyses. Collections of hypothetical causes that are highly concordant with the observed expression profiles are the output of causal analysis and form the inferred mechanism of regulation.

Before the model is used to infer mechanisms of regulation, the model is first probed to determine the competency of the model to do reasoning in the modeled system. For example, the exemplary model of human skeletal muscle biology and DM2 was probed to determine its ability to do reasoning in the muscle. Competency was assessed by introducing a set of perturbations and then using forward causal analysis to predict the downstream effects caused by such perturbations. Table 1 details a few of the numerous perturbations that were used to test the competency of the human skeletal muscle biology and DM2 model.

TABLE 1
Assessment of Causal Model Competence for
Human Skeletal Muscle Biopsy and DM2

| | | Prediction Via Causal |
|---|---|---|
| Perturbation | Mechanism in Model | Analysis |
| Effect of PTP1B | PTP1B dephosphorylates | Increased insulin |
| inhibitor on | insulin receptor; PTP1B | receptor activation |
| insulin signaling | inhibition increases | and increased insulin |
| | insulin receptor | signaling |
| | signaling | |
| Effect of thiazo- | Increase tyrosine | Increased insulin |
| lidinediones | phosphorylation of Cbl, | signaling |
| mediated through | which activates Cbl- | |
| PI3K kinase | dependent PI3K kinase | |
| activity | activity and its | |
| | downstream signaling | |
| Effect of inac- | AMP-kinase stimulates | Decreased exercise- |
| tivation of AMP- | glucose import | stimulated glucose |
| kinase on exercise- | | import |
| stimulated glucose | | |
| import | | |
| Effect of MEK | LY492002 inhibits ETS2 | Decreased ETS2 |
| inhibitor LY492002 | activation, and PTEN | transcriptional |
| or PTEN overex- | phosphatase activity | activity |
| pression on ETS2 | decreases ETS2 activity | |
| transcriptional | through inhibition of | |
| activity | ETS2 phosphorylation | |
| Effect of p85a | Increased of AKT | Increased AKT |
| knockout on insulin | abundance via inhibition | phosphorylation and |
| signaling as | of PI3K kinase activity | kinase activity |
| measured by AKT | | |
| kinase activity | | |
| Effect of GSK3B | GSK3B inhibits insulin | Increased insulin- |
| inhibitor on | stimulated glucose | stimulated glucose |
| insulin-stimulated | transport | transport |
| glucose transport | | |
| Effect of increase | TNF inhibits IRS1 | Inhibition of insulin |
| in TNF protein | binding by promoting | signaling |
| abundance on | its serine phosphor- | |
| insulin signaling | ylation | |
| Effect of ENPP1 | ENPP1 dephosphorylates | Inhibition of insulin |
| phosphatase | insulin receptor | signaling |
| activity on | | |
| insulin signaling | | |

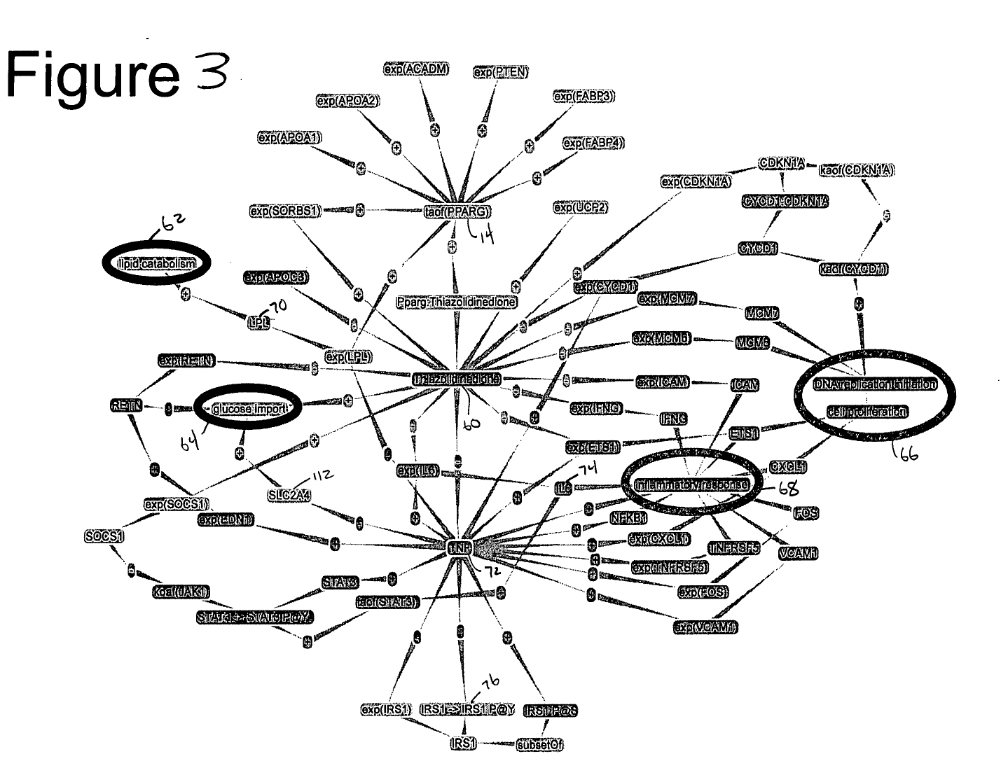

One exemplary perturbation of the human skeletal muscle and DM2 model, the effect of thiazolidinediones mediated through PI3K kinase activity, is represented as a directed graph in FIG. 3. FIG. 3 is an illustrative representation of an in silico prediction of the mechanism and efficacy of the insulin-sensitizing thiazolidinedione class of drugs 60 and the resulting downstream biological effects. FIG. 3 illustrates that the muscle model, through forward causal analysis, predicts an increase in lipid catabolism 62 and glucose import 64 as well as a decrease in cell proliferation 66 and a decrease in the inflammation response 68. With reference to FIG. 3, those activities that are predicted to increase following introduction of thiazolidinedione are lightly shaded and those activities that are predicted to decreases are darkly shaded. The muscle model accurately predicts enhanced insulin sensitivity (not shown) via IRS1 tyrosine phoshorylation 76, increased glucose import 64 via increased GLUT4 expression (not shown) and decreased SLC2A4 abundance 112, decreased cytokine-induced insulin resistance 68 via decreased TNFa abundance 72 and expressed IL-6 74, and enhanced lipid catabolism 62, via increased abundance of expressed LPL 70 caused by increased transcriptional activity of PPARG 14.

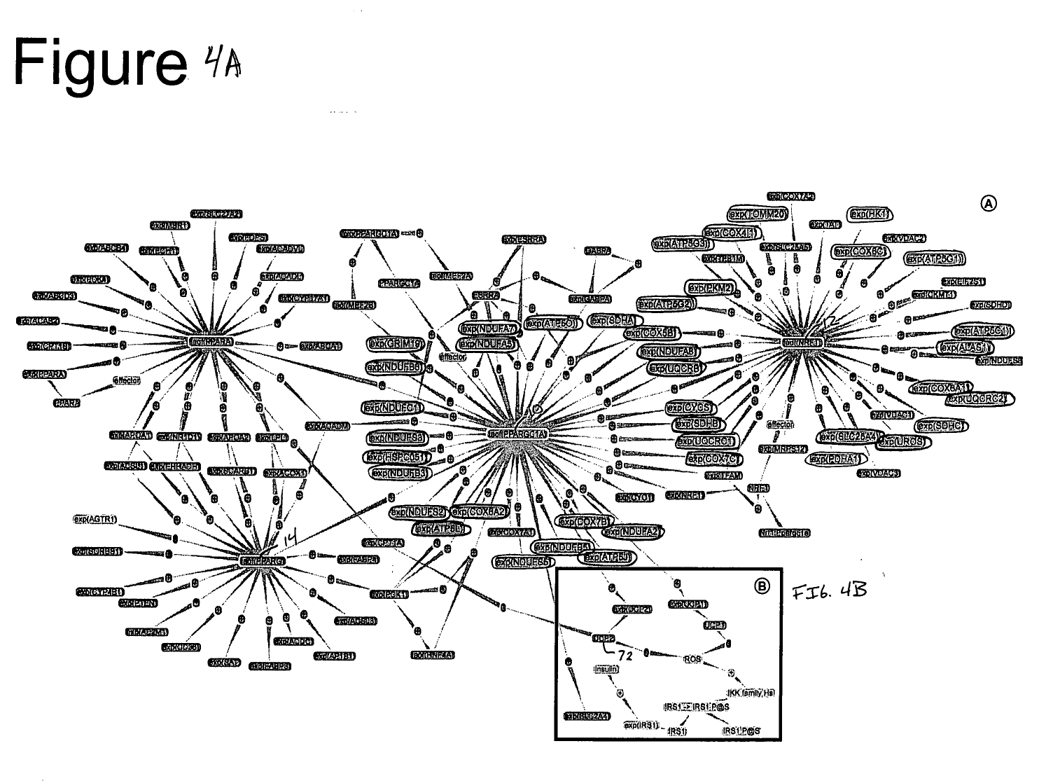

The competence of the model to discover the transcriptional network of the OXPHOS genes is shown in FIG. 4A. FIG. 4A is an illustrative representation of a forward causal analysis within the muscle model following a perturbation to decrease the transcriptional activity of PPARGC1a 10. Because modulation of the OXPHOS genes is thought to be predicated on decreases in the transcriptional activity of the co-activator PPARGC1a 10, forward causal analysis was performed using PPARGC1a 10 as a starting point. With reference to the directed graph of FIG. 4A, those activities that are predicted to increase following decreased transcriptional activity of PPARGC1 a 10 are lightly shaded and those activities that are predicted to decrease are darkly shaded. The expression of genes actually observed to change in the studies are both encircled and darkly shaded. The causal analysis confirmed that a decrease in PPARGC1a 10 transcriptional activity and consequently a decrease in NRF1 12 transcriptional activity could explain the observed changes in expression profiles of OXPHOS genes from both of the studies. Furthermore, the analysis outlined numerous plausible mechanisms by which decreased transcriptional activity of PPARGC1a 10 could cause a decrease in insulin sensitivity and glucose import in muscle.

FIG. 4B is an illustrative representation of an exemplary mechanism linking a decrease in OXPHOS expression to a decrease in insulin sensitivity and glucose import in muscle. The directed graph of FIG. 4B highlights an exemplary predicted mechanism that can be mediated directly through decreased GLUT4 expression or indirectly through decreased UCP2 72 expression, one of the many mechanisms linking a decrease in OXPHOS expression to a decrease in insulin sensitivity and glucose import in muscle.

Predicted Regulatory Mechanism of Calcineurin on Post-Transplantation Diabetes Mellitus

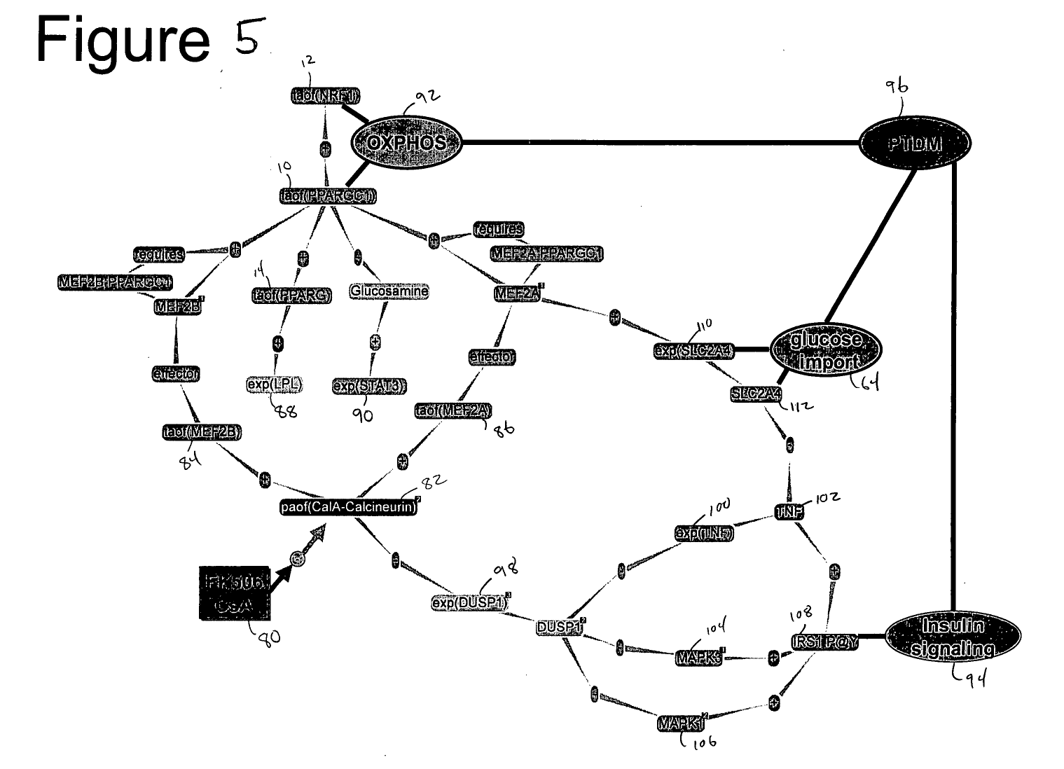

The exemplary causal model contains transcriptional control information for approximately 56% of genes that were modulated combined across the modeled studies, including 101 of the 221 modulated genes in the Scandinavian study and 30 of the 77 modulated genes in the Mexican-American study. Reverse causal analyses were performed on each study separately, and regulatory mechanisms were scored and ranked based on concordance between predicted and observed expression changes. Intersection of the causal analyses across the two studies revealed a predicted regulatory mechanism that is highly significant according to the richness and concordance metrics and can explain 49 of the 131 gene expression changes known to the model, detailed in Table 2 and FIG. 5.

Table 2 outlines the hypothetical regulatory mechanisms present in both studies, the number of predicted changes that matched each observed direction of change, and the prediction's richness and concordance values. With reference to Table 2, a causal hypothesis is a change in abundance or activity of a component in the model that would cause observable changes in other components in the model. The possible column refers to the number of expression changes downstream of the causal hypothesis for which the model can make predictions. The correct column refers to the number of predicted changes that matched the observed direction of change for a given causal hypothesis. The contradiction column refers to the number of predicted changes that do not match the observed direction of change for a given causal hypothesis. The conflict column refers to the number of expression changes ambiguously determined from the model for a given causal hypothesis. As previously discussed, richness is the probability that the number of observed changes in a given model could have occurred by chance along for a given causal hypothesis, and concordance is the probability that the number of observed changes that match the directionality of the changes predicted by the model could have occurred by chance alone for a given causal hypothesis.

TABLE 2
Hypothetical Regulatory Mechanisms Present in Both Studies

| | Change in activity | Predictions | | | | P values | |
|---|---|---|---|---|---|---|---|
| Causal Hypothesis | or abundance | Possible | Correct | Contradiction | Conflict | Richness | Concordance |
| taof(PPARGC1A) | Decrease | 35 | 25 | 0 | 0 | 2.4E−06 | 9.8E−04 |
| taof(NRF1) | Decrease | 36 | 22 | 2 | 0 | 2.5E−02 | 1.9E−01 |
| taof(MEF2 family Hs) | Decrease | 71 | 47 | 2 | 0 | 1.6E−05 | 1.7E−03 |
| paof(CalA-Calcineurin family Hs) | Decrease | 62 | 49 | 3 | 0 | 3.3E−06 | 1.7E−03 |

FIG. 5 is an illustrative embodiment of the intersection of the causal analyses of treatment with the calcineurin inhibitors tacrolimus (FK506) and cyclosporine A (CsA) across the two studies. With reference to the directed graph of FIG. 5, a common regulatory mechanism can be linked to post-transplant diabetes mellitus (PTDM) 96 after treatment with the calcineurin inhibitors tacrolimus (FK506) or cyclosporin A (CsA) 80. The common regulatory mechanism indicates that introduction of a calcineurin inhibitor, such as tacrolimus (FK506) or cyclosporin A (CsA) 80, decreases the phosphatase activity of CalA-Calcineurin 82, thereby decreasing calcineurin signaling within the system. Calcineurin signaling is linked to the regulation of the OXPHOS genes NRF1 12 and PPARGC1 10, the regulation of glucose import 64, and the regulation of insulin signaling 94, all of which are associated with post-transplant diabetes mellitus 96.

With reference to FIG. 5, decreased calcineurin signaling 82 is linked to increased transcriptional activity of both MEF2A 86 and MEF2B 84, which have been shown, along with other necessary factors, to increase the transcriptional activity of PPARGC1a 10. Increased transcriptional activity of PPARGC1a 10, one of the OXPHOS gene, is consistent with an increase in the transcriptional activity of another OXPHOS gene, NRF1 12. Moreover, an increase in PPARGC1a 10 transcriptional activity, and therefore an increase in PPARGC1a protein abundance (not shown), is consistent with observed increases in the expression levels of STAT3 90 and LPL 88 in both studies.

Still referring to FIG. 5, decreased calcineurin signaling 82 is also linked to a decrease in the level of expression of the DUSP1 gene 98, which in turn is related to decreased expression of TNF 100 and also decreased activity of MAPK1 106 and MAPK3 104. Both decreased expression of TNF 100 and decreased activity of MAPK1 106 and MAPK3 104 are linked to decreased IRS1 tyrosine phosphorylation 108, which is implicated in insulin signaling 94 and post-transplant diabetes mellitus 96.

With continued reference to FIG. 5, decreased calcineurin signaling 82 is also related to glucose import through two different regulatory mechanisms. First, decreased calcineurin signaling 82 increases the transcriptional activity of MEF2A 86, which is associated with increased expression of SLC2A4 110, which is in turn implicated in glucose import 64. Second, decreased calcineurin signaling 82 is related to decreased expression of DUSP1 98, which decreases the expression level of TNF 100. Decreased abundance of TNF 102 results in a decreased abundance of SLC2A4 112, which is also implicated in glucose import 64 and post-transplant diabetes mellitus 96.

Assessment and Treatment of Post-Transplantation Diabetes Mellitus

The invention provides a theoretical framework to support further assessment of skeletal muscle calcineurin expression by transcript profiling or proteomic methods independent of other predisposing factors. The calcineurin inhibitors tacrolimus (FK506) and cyclosporine A (CsA) are essential immunosuppressive drugs for the clinical management of rejection in organ transplantation. While calcineurin is known to be widely distributed throughout the body, including the brain, heart, liver, kidney, pancreas and skeletal muscle, the rationale for using calcineurin in transplant rejection protocols has been the targeting of leukocyte-associated calcineurin as a means to suppress leukocyte function and prolong graft survival. However, immunosuppressive therapy with these inhibitors represents a significant independent risk factor for the development of post-transplant diabetes mellitus, and post-transplant diabetes mellitus significantly compromises graft and patient survival.

Therefore, one aspect of the invention is a method of developing an immunosuppressant drug with reduced propensity to induce DM2 symptoms by assaying candidate molecular entities for binding preferentially to leukocyte isoform calcineurin and less preferentially to muscle cell isoform calcineurin. It has been shown that immunosuppressant drugs that bind to leukocyte isoform calcineurin have a reduced propensity to induce DM2 symptoms, whereas immunosuppressant drugs that bind to muscle cell isoform calcineurin have a greater propensity to induce DM2 symptoms. Therefore, the identification of a molecular structure that competitively binds to leukocyte isoform calcineurin over muscle cell isoform calcineurin holds the promise of providing the benefits of immune suppression therapy with reduced risk of developing DM2.

Calcineurin isoforms can be in the form of dimers of one or more type of subunit, including but not limited to a catalytic A subunit and a calcium-binding B subunit. The dimers may be in a variety of forms including, for example, A-A, A-B, or B-B calcineurin dimers. Moreover, the leukocyte isoform calcineurins and muscle cell isoform calcineurins can be splice variants of each other.

According to one embodiment, candidate molecular entities assayed in the method are selected for their proximity in chemical space to the calcineurin inhibitor tacrolimus (FK506) or a cyclosporine, including by not limited to cyclosporine A (CsA). Molecular entities are proximate in chemical space if they are similar in three-dimensional form or have a similar chemical activity as the known chemical entity. According to another embodiment, candidate molecular entities are selected for their known ability to target a particular characteristic of calcineurin, for example, its phosphatase activity. Candidate molecular entities may be natural molecules or synthetic molecules developed using combinatorial chemistry or other suitable techniques.

According to another embodiment, the binding assay is conducted using immobilized calcineurin isoforms or labeled soluble calcineurin isoforms. The binding assay can be a screening assay conducted against a molecular library of potential candidate molecules. Differential binding of the candidate molecules to the calcineurin isoforms for muscle and leukocytes is then determined. The binding assay can be conducted using any appropriate screening method known in the field. In one embodiment the screening method is a binding assay. In one embodiment of this assay the calcineurin isoform against which the candidate drug is being tested is bound to a substrate and the bound calcineurin exposed to the drug candidate. The substrate is then washed to remove any non-bound drug candidate and the amount of bound drug candidate measured. In various embodiments the measurement of the bound drug candidate is accomplished radioactively or fluorescently, to name but two methods of detection. In each respective case the candidate drug molecule is previously labeled with a radioactive isotope or a fluorophore, respectively.

If the radioisotope method is used, once the unbound drug is washed off the substrate, the substrate can be measured as a whole radiographically or by any other method known to one of ordinary skill in the art. If the fluorophore method is used, once the unbound drug candidate is washed from the substrate, the substrate can be interrogated using light of the proper frequency so as to cause any bound drug to fluoresce. Then by viewing the substrate through the appropriate filter to eliminate the excitation light, any bound drug can be easily detected. Because the amount of radioactivity detected or the amount of fluorescence measured is a function of the amount of bound drug, the relative affinity for various drugs to the various calcineurin isoforms can be determined.

One attribute of a substrate assay as just described is that multiple calcineurin isoforms located on different locations on the substrate can be tested at the same time against a candidate drug. Although this embodiment is described as the calcineurin isoform being bound and the candidate drug being labeled, it is possible to reverse the order and bind a plurality of candidate drugs to the substrate and expose them to various unbound calcineurin isoforms, each properly labeled with a radioactive isotope or a fluorophore.

If the calcinuerin isoform is used to challenge the bound drug, the presence of unlabeled calcineurin isoform bound to the substrate-bound drug, can be detected also using labeled antibodies to the calcineurin isoform. In various embodiments the labeled antibodies are labeled with a radioisotope or labeled with a fluorophore. That is, in this embodiment, the unlabeled calcineurin isoform is allowed to bind to the bound drug, and the substrate then washed to remove any unbound calcineurin isoform. Labeled antibodies to the calcineurin isoform are then added to the substrate and allowed to bind to the calcinerin isoform. The unbound labeled antibodies are then washed from the substrate and the bound antibodies measured as discussed previously.

In addition, if a different fluorophore or radioisotope is used for each calcineurin isoforms, all the calcineurin isoforms of interest can be exposed to the bound drug prior to any detection taking place, because the various bound calcineurin isoforms can be separately detected. In addition, although the embodiments just described utilize a substrate with bound reactants, the same process can be used with an affinity column with one of the reactants bound to the particles of the column.

Competitive binding can also be used to determine the preferential binding of the calcineurin isoform to the drug. In this type of assay a drug candidate is mixed with multiple isoforms of calcineurin and allowed to bind. Typically there is an excess of calcineurin isoforms in the mixture. In one embodiment each calcineurin isoform is separately labeled with a distinctive marker. Once the drug and calcineurin isoforms are allowed to bind, the mixture is placed on a separation column, an electrophoretic gel, or in a centrifugal gradient in a centrifuge and allowed to separate by their differences in size, size and charge, and density, respectively. Because the unbound drug and unbound calcineurin will migrate in each of the devices differently from the bound drug and calcineurin, one can easily determine which calcineurin isoform has the higher affinity for the candidate drug.

In addition to determining the binding preferences of candidate immunosuppressant drugs the invention puts on a secure theoretical footing a method for determining the onset, severity, progression or response to treatment of post-transplant diabetes mellitus. In this method, a patient at risk of contracting post-transplant diabetes mellitus has a biopsy of muscle tissue and the expression or activity of the following proteins down-stream from the calcineurin target measured: HDAC5, HNF4A, NRF1, PPARGC1, PPP3CA, and calcineurin proteins. The activity of these enzymes can be determined by measuring reaction products which occur when each is placed in the presence of its respective substrates. The expression levels of the proteins can be determined through the quantitative measurements using specific antibodies or protein separation techniques followed by colorimetric measurements. In addition expression measurements of the nucleic acids extracted from the biopsy material can provide a quantitative measurement of the amounts of enzymes produced.

Once these quantitative measurements are made a change of activity or expression as shown below in at least two of the enzymes is indicative of the patient developing post-transplantation diabetes mellitus: increased expression or activity of HDAC5 protein; increased expression or activity of HNF4A protein; decreased expression or activity of NRF1 protein; decreased expression or activity of PPARGC1 protein; decreased expression or activity of PPP3CA protein; and decreased expression or activity of calcineurin proteins. This method can be used to determine: onset, severity, progression or response to treatment of post-transplantation diabetes mellitus.

Assessments of skeletal muscle calcineurin subunits, precursors, or proteins known to be affected downstream may be used to, among other things, predict the development of new-onset diabetes post-transplantation, screen disease progression and conversion of a pre-diabetic to an overt diabetic state during the course of immunosupressive therapy, therapeutically monitor the pharmacologic effects of calcineurin inhibition as a surrogate of glucose tolerance, and design individualized pre-transplant and post-transplant immunosuppressive protocols to minimize drug-induced new-onset diabetes.

The teachings of the patents and non-patent publications discussed or referenced herein are incorporated by reference in their entirety. Variations, modifications, and other implementations of what is described herein will occur to those of ordinary skill in the art without departing from the spirit and the scope of the invention as claimed. Accordingly, the invention is not to be defined by the preceding illustrative description but instead by the spirit and scope of the following claims.

What we claim is:

1. A method of developing an immunosuppressant drug with reduced propensity to induce type II diabetes symptoms, the method comprising:
assaying candidate molecular entities for binding preferentially to leukocyte isoform calcineurin and less preferentially to muscle cell isoform calcineurin.

2. The method of claim 1 wherein the candidate molecular entities comprise molecules adjacent in chemical space to FK506 or a cyclosporine.

3. The method of claim 1 wherein the binding assay is conducted using immobilized calcineurin isoforms or labeled soluble calcineurin isoforms.

4. The method of claim 1 wherein the leukocyte isoform calcineurin is a calcineurin dimer.

5. The method of claim 1 wherein the muscle cell isoform calcineurin is a calcineurin dimer.

6. The method of claim 1 wherein the leukocyte isoform calcineurin and the muscle cell isoform calcineurin are splice variants of each other.

7. A method for determining the onset, severity, progression or response to treatment of post-transplant diabetes mellitus comprising determining from a patient at risk of contracting post-transplant diabetes mellitus, at least two of:
increased expression or activity of HDAC5 protein;
increased expression or activity of HNF4A protein;
decreased expression or activity of NRF1 protein;
decreased expression or activity of PPARGC1 protein;
decreased expression or activity of PPP3CA protein; and
decreased expression or activity of calcineurin proteins.

8. The method of claim 7 comprising determining decreased expression or activity of PPARGC1 protein.

9. The method of claim 7 comprising assaying for protein concentrations or activity in a muscle cell from a muscle biopsy from a said patient.

10. The method of claim 7 comprising assaying for gene transcripts or biomolecules produced by interaction with a said protein as a proxy for an increase or decrease in said protein expression or activity.
